(19) United States
(12) Patent Application Publication
(10) Pub. No.: US 2007/0248661 A1
(43) Pub. Date: Oct. 25, 2007

(54) Capsule for Releasing Agents Contained Therein at Defined Points In a Body

(75) Inventors: Wilfried Andrä, Jena (DE); Matthias Erich Bellemann, Jena (DE); Henri Danan, Strasbourg (FR); Silvio Dutz, Meura (DE); Stefan Liebisch, Hartmannsdorf (DE)

Correspondence Address: JORDAN AND HAMBURG LLP, 122 EAST 42ND STREET, SUITE 4000, NEW YORK, NY, 10168, US

(73) Assignee: FACHHOCHSCHULE JENA, Jena (DE)

(21) Appl. No.: 11/632,200

(22) PCT Filed: Jun. 15, 2005
(86) PCT No.: PCT/DE05/01086

§ 371 Date: Jan. 16, 2007

(30) Foreign Application Priority Data
Jul. 13, 2004 (DE) DE 10 2004 034 35

(51) Int. Cl. A61K 9/48

(52) U.S. Cl. 424/452

(57) ABSTRACT

A capsule is capable of releasing at least one agent that is contained therein by heating at least one heating element under the effect of at least one alternating magnetic field at a defined point in a body, said capsule dissolving when entering in contact with a dissolving liquid. The capsule avoids getting stuck on stenoses while being thermally acceptable and favorable from the point of view of energy. In the capsule, the heating element is at least partially surrounded with a capsule part, material of which is provided with greater thermal resistance than the walls of common capsules used for medicaments.

### BACKGROUND OF THE INVENTION

The invention relates to a capsule for releasing agents contained therein at defined points in a body, particularly useful for the examination of the digestive tract.

It is a generally known fact that a significant part of all drugs is taken in the form of tablets or capsules containing agents that can be absorbed in the digestive tract. With the exception of the stomach, for which the application of medicaments is well managed, the precise location of the absorption could not be adjusted until now. An inherent disadvantage resides in the fact that the passage speed through the intestine and the pH value in the intestine varies considerably for different persons and even for a particular individual depending upon his/her condition. Therefore, even especially prepared capsules used for medicaments, e.g. time controlled, enzyme controlled, pH value controlled or pressure controlled capsules, imply the risk that the agent may pass the target area without being absorbed in a sufficiently large quantity. But, conversely, if intentional overdoses are used, there will be the risk of unintended side effects.

In the past, a number of methods, arrangements and capsules have become known that were focused on the determination of the specific position of a medicament capsule in the intestine and, if the target position was reached, were implemented to release the agent via remote control; see Andrä, W. et al., A novel method for real-time magnetic marker monitoring in the gastrointestinal tract, Physics in Medicine and Biology 45: 3081-3093 (2000); Hemmati, A., The Site of Iron Absorption in the Gastrointestinal Tract, German Med. Mth., Vol. XIII: 569-573 (1968); DE 29 28 477 A1; Grönig, R., Computer-controlled drug release from small-sized dosage forms, Journal of Controlled Release 48: 185-193 (1997); U.S. Pat. No. 510,801 A; DE 19745 890 A1; U.S. Pat. No. 4,239,040 A; U.S. Pat. No. 5,279,607 A. Most of the capsules described in the aforementioned publications have at least one of the following disadvantages. First, they contain a hard cover. Thus, there is the risk that such capsules may get stuck on stenoses in the intestine and possibly have to be removed by operative surgery. According to the publication by Rösch, T et al., in Derzeitige klinische Indikationen der Kapsel-Endoskoopie (Current clinic indications of capsule endoskopy) in the German journal Zeitschrift für Gastroenterologie (Journal for gastroenterology) 40: 971-978 (2002), this danger can even exist if stenoses have not been registered during a previous x-ray examination. Additionally, the capsules mentioned include hard parts, such as metal springs, batteries and electronic components or circuits that can have a toxic effect if they contact the intestinal wall.

These two aforementioned disadvantages can be avoided by means of the intestine therapy capsule manufactured according to DE 197 45 890 A1 and, in the same way, by an already suggested capsule with a rotating ball, if suitable substances are used. But these solutions suggest a disadvantage that is due to the mechanism of release. The release is achieved by heating up a partial volume of the capsule (hereinafter referred to as heating element) in an alternate magnetic field by magnetic losses or by friction losses to such a degree that an organic substance melts or the opening of the capsule is activated in another way. Here, the intestine content or the intestinal wall, or the liquid agent is positioned in the direct vicinity of the heating element. The thermal conductivity of this environment is so high that the increased heating of the heating element causes the dissipation of an increased amount of heat into the environment that therefore does not contribute to the temperature rise of the heating element.

The maximally achievable rise in temperature is determined by the fact that the total power that is input by the alternating field is dissipated into the environment. According to the theory of thermal conduction, the maximally achievable rise in temperature is proportional to the input power and approximately reversely proportional to the thermal conductivity of the environment. The thermal conductivity of the environment for the capsules described is 0.2 W/(m-K) or higher. The thermal resistance between the heating element and the environment is on the order of 1 to 10 K/W. The selected input power of the alternate magnetic field must be sufficiently high to reach the desired maximum temperature despite the heat dissipation. The input power of the alternate magnetic field must not be as high as may be desired because, otherwise, an excessive heating of the patient can be caused by eddy current losses in the body tissue [Brezovich, I. A., Low frequency hyperthermia: capacitive And ferromagnetic thermoseed methods, Medical Physics Monographs 16: 82-111 (1988)].

It is therefore the object of the present invention to avoid the described disadvantages in an capsule design in accordance with the invention directed to preventing the capsule from getting stuck on stenoses while endowing the same with thermally acceptable characteristics and favorable properties from the point of view of energy.

### SUMMARY OF THE INVENTION

The object of the present invention is achieved by a capsule for releasing at least one agent contained therein at defined positions in a body, which comprises capsule parts enclosing the capsule including at least one insulating capsule part, a material of which has a greater thermal resistance than an other of said capsule parts. At least one heating element at least partially surrounded by said at least one insulating capsule part is provided, wherein the capsule is openable by heating the at least one heating element under an effect of at least one alternating magnetic field. The capsule is dissolvable when entering in contact with a solving liquid. The thermal resistance of the capsule part(s) surrounding the heating elements(s) should be higher than the thermal resistance of the other capsule parts or of common capsules used for medicaments, at least by one order. The invention makes it possible that, on the one hand, all parts of the capsule consist of substances that disintegrate or dissolve when entering in contact with a liquid medium and, on the other hand, the capsule part designated as the heating element is surrounded by a cover that has a considerably greater thermal resistance than 10 K/W. The power required to reach the release temperature is reduced by adding a thermal insulation envelope. The heating generated under the influence of the alternate magnetic fields in at least one part of the capsule leads to a remote-controlled evaporation of an easily evaporating liquid disponed threrin. This liquid presses the agent (or several agents) out of the interior of the capsule, or it causes the capsule wall, which is comprised of parts, to burst. An advantageous embodiment of this invention contains a capsule part at least partially surrounding the heating element, which is closed against the agent by a wall, variable with respect to its position and/or its expansion.

In a preferred embodiment, at least one heating element that contains a magnetic powder, e.g. Fe₃O₄ (magnetite), is surrounded by a thermally insulating envelope. Said envelope can be double-walled, in which the walls consist essentially of water-soluble material, such as hard gelatin or sugar, and which are separated by a gas layer, e.g. air. As the whole capsule may only have a specific size to avoid difficulties when swallowing it, the thickness of this envelope is also reduced. Therefore, this thickness must be considered when comparing the thermal conductivity without and with this envelope. Ideally, the thermal resistance of the envelope is about 500 K/W, in relation to a normal medicament capsule of the same size of which the thermal resistance is about 10 K/W. Instead of the double-walled envelope it is also possible to use a porous envelope of water-soluble material with enclosed gas pockets. The thermal conductivity of such porous materials and the conductivity of air differ only slightly.

The present invention will now be described in more detail by way of the following schematic examples.

### BRIEF DESCRIPTION OF THE DRAWINGS

FIG. 1 is a longitudinal section through capsule in accordance with an embodiment of the invention with a bag that can be bloated;

FIG. 2 is a longitudinal section through an inventive capsule with a stamp;

FIG. 3 is a longitudinal section through an inventive capsule with a movable dividing wall; and

FIG. 4 is a longitudinal section through an inventive capsule with two heating elements and two dividing walls that can change their position.

### DETAILED DESCRIPTION OF THE INVENTION

FIG. 1 depicts a capsule 10, which comprises two parts 11, 12 with a common geometric axis X-X that are hermetically connected with each other, nested, and for example made of hard gelatin. The open end of the bigger part 11 slides over the smaller part 12 and, in a two-dimensional bent area, the bigger part 11 being provided with a small opening 112 that is closed by a membrane 13 against the spontaneous escape of the agent 14 contained in the capsule 10. Normally, the agent does not contain water. Alternative to use of a membrane 13 closure, the opening 112 can be made sufficiently small or have a valve design such that the spontaneous escape of the agent 14 is not possible under normal pressure conditions. The capsule part 12 is double-walled and provided with air or another suitable insulation material between the walls for purposes of heat insulation 15. Capsule part 12 contains a thin-wall bag 17 made of latex or polyethylene and filled with a heating element 16. The folded structure 171 of this bag 17 allows the enlargement of the volume of the heating element 16 by at least 1 cm³ or to double the volume. In the example, the heating element 16 is made of a composition of about 40 Vol. % Fe₃O₄, the losses of which are about 1 J/kg in a magnetic reversal cycle, and of about 60 Vol-% ethyl alcohol that has a boiling point at 78° C. and evaporates at this temperature and its expansion generates a pressure in the capsule 10 that opens the opening 112 so that the agent 14 escapes to the outside. It is also possible to use another easily evaporating and biocompatible liquid instead of ethanol. The rise in temperature is caused by an alternate magnetic field, which is generated by an electric coil 18 in a commonly known manner, in combination with the Fe₃O₄components in the bag 17.

A thin layer (eg., film or foil) 19 of polyethylene, shellac or another suitable substance covering the whole capsule 10 protects the capsule 10 against decomposition in a water containing and/or enzyme containing environment, that is given, for example, in the intestinal tract. The opening 112 can also be arranged at another point of the capsule part 11. Instead of the double-walled part 12, it is also possible to use a component that contains gas pockets and is made of water-soluble material. Finally, the capsule 10 is neither bound to the embodiment shown in FIG. 1 nor to the two-part design described with respect thereto. The position of the coil 18 is schematically illustrated, and is located outside the body during the application.

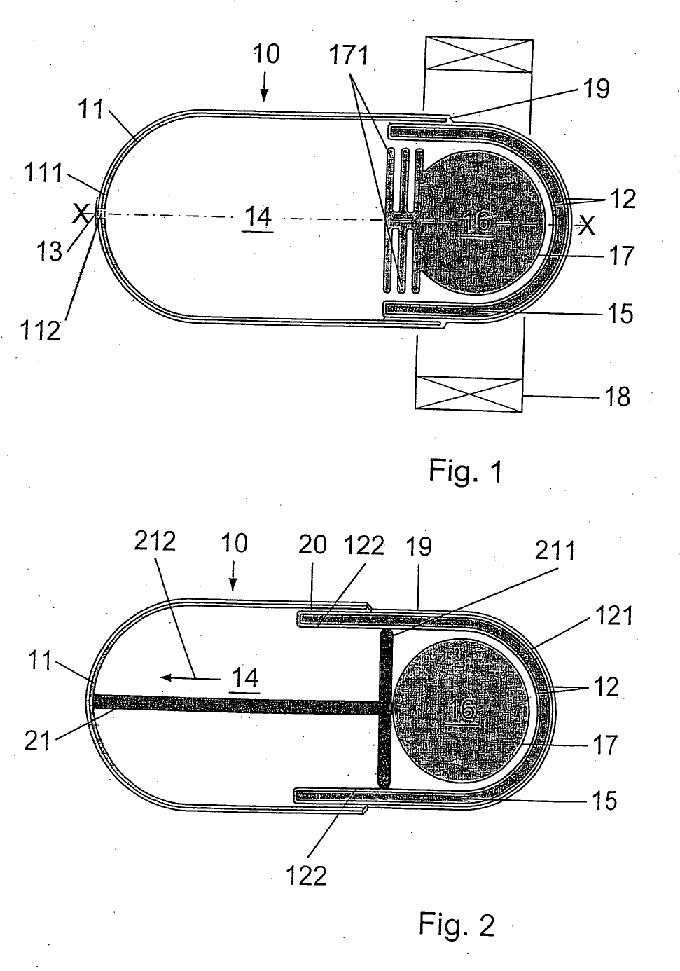

FIG. 2 also depicts a capsule 10, the parts 11 and 12 of which are held together by a foil (film) 19 that surrounds the entirety of capsule 10, and which should not be between the nested parts, i.e., in the area 20. In this area, a biocompatible lubricant, e.g. paraffin, can optionally be added between the two capsule parts 11, 12. The heat-insulating part 12 includes bag 17 therein with heating element 16 that is located in the direct vicinity to the bent area 121 of part 12 and supports itself against the bent area 121 during the expansion of the heating element 16. Additionally, the part 12 is provided with guide surfaces 122 for the supporting and sealing elements 211 of a stamp 21 that can move towards the axial direction marked by an arrow 212. If the heating element 16 is heated in the manner described with reference to FIG. 1, the liquid in the heating element expands and presses the stamp 21 with the part 11 towards the direction indicated by the arrow 212. The foil 19 is torn in the area 20 and the agent 14 is discharged to the outside, for example into the intestinal tract. The penetrating water or other liquid decomposes the parts 11 and 12 from the inside. Like the supporting elements 211, the stamp 21 should be made of a water-soluble material.

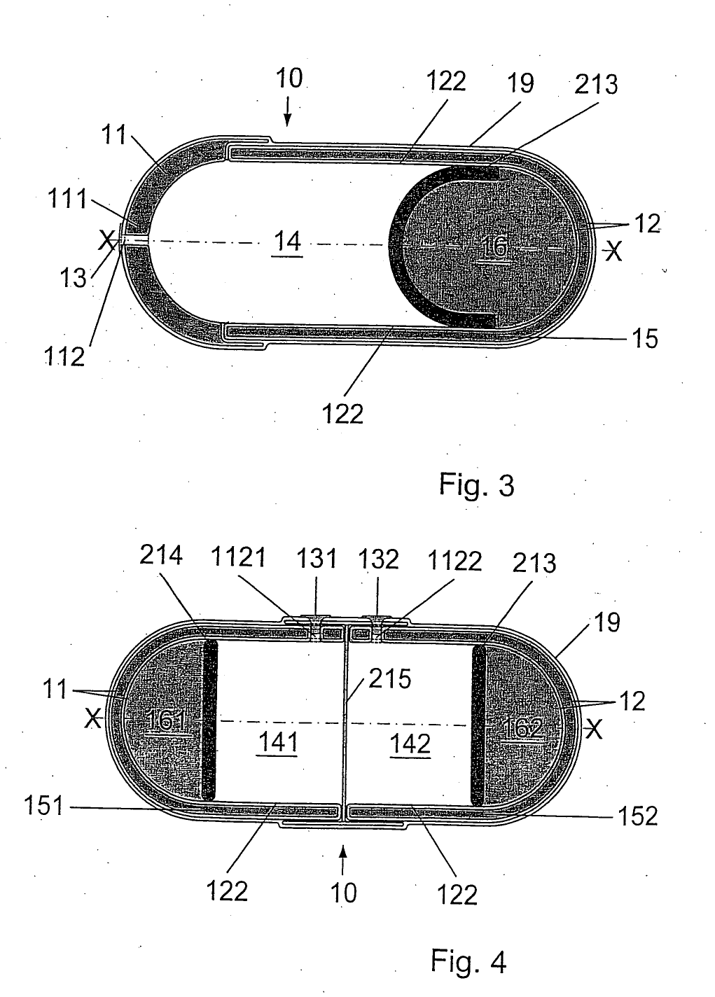

FIG. 3 again depicts a two-part capsule 10 with a part 12 that is filled with an insulation material 15 and which is slid axially into the part 11 that is provided with an opening 112. Here, the part 12 is fixed by a foil 19. Part 12 contains (preferentially without the bag 17 of FIG. 2) an element 16 that acts like a heating element if an alternate magnetic field is applied from the outside. A movable diving wall 213 separates said element 16 from the agent 14 which takes up most of the part of the capsule interior. When heating the element 16, element 16 expands, for example, to double the volume and presses onto the agent 14 in such a way that a membrane 13 that closes the opening 112 in the capsule part 11 is caused to burst. The agent 14 can escape via the opening 112 and the solvent, for all capsule parts including the dividing wall 213, can flow in. Furthermore, the disclosure relative to FIGS. 1 and 2 applies analogously to FIG. 3.

In is noted that the dividing wall 213 that is movable in the capsule part 12 is not bound to the configuration shown in FIG. 3.

FIG. 4 also includes a two-part capsule with the two adjoining parts 11 and 12 that are provided with insulation materials 151, 152, and which therefore exhibit a greater thermal resistance than the other parts of the capsule. Each of the parts 11 and 12 contain elements 161 and 162 each that functions like a heating element if an alternate magnetic field is applied from the outside and which is separated from the agent 141 and 142 by a movable and/or expandable dividing wall 213 and 214. If the corresponding elements are heated, they expand to double the volume for example and press the agents, possibly one after the other, to the outside through the openings 1121, 1122 that can be closed for example by a plug (a valve) 131, 132. The heating elements 161 and 162 can be comprised of different materials or different compositions of easily evaporating liquids and magnetic oxide powders so that the evaporation takes place at different temperatures or for different power values of the alternate magnetic field. Furthermore, the agents 141 and 142 in the capsule parts 11, 12 can be different. These agents 141, 142 can also be prepared in such a way that the desired effect is only produced after their mixture. Within the capsule 10, the unintended mixture of the two agents 141 and 142 can be avoided by a fixed dividing wall 215.

The described invention demonstrates advantage in comparison to the state of the art. Due to the much greater thermal resistance of the insulation of part 12, the power of the alternate magnetic field supplied to the capsule 10 can be considerably less for the same intended maximum temperature, e.g. 78° C., than for the capsules without an insulation cover. Although the volume of the heating element becomes smaller due to the insulation, the required supplied power for the same maximum temperature is ideally less than 1% of the power that must be supplied for the capsule without thermal insulation. Another advantage is offered by the expandable bag 17 or a flexible and/or movable wall that closes the heating element 16 against the agent 14. Such approach avoids the use of pistons and similar elements made of hard material. When heating the magnetic powder above the temperature of ebullition of the liquid contained in the heating element, said liquid will evaporate and the agent will be discharged after a short period of time. Thus, the point of time and the location of the agent application are much better defined in this invention than in the methods and arrangements known heretofore. It is even possible to apply the agent subsequently in several portions if the supplied alternate field power is measured out appropriately. The construction of the capsule 10 has the effect that after the discharge of the agent 14, the water-containing intestinal liquid, for example, enters into the capsule 10 and decomposes the capsule parts 11, 12 or the hard gelatin stamp 21 from the inside. The other parts of the capsule (magnetic powder, polyethylene foil) can be easily ducted so that the remainder of the capsule do not get stuck on stenoses.

The individual features or any combination thereof described in the invention and the figures are inclusive of, but not limiting of, the invention, which is defined by the claims.

### LIST OF REFERENCE NUMERALS

10 capsule

11, 12 (capsule) parts

13 membrane

14, 141, 142 agents

15, 151, 152 air, insulation material

16, 161, 162 heating elements

17 bag

18 coil

19 layer, foil

20 area

21 stamp

111,121 bent zones

112, 1121, 1122 openings

122 guide surfaces

131, 132 valves, plugs

171 folded structure

211 sealing and supporting elements

212 arrow

213, 214 flexible and/or movable dividing walls

215 fixed dividing wall

X-X axis

1-19. (canceled)

20. A capsule for releasing at least one agent contained therein at defined positions in a body, comprising:
capsule parts enclosing said at least one agent in said capsule including at least one insulating capsule part, a material of which has a greater thermal resistance than an other of said capsule parts; and
at least one heating element at least partially surrounded by said at least one insulating capsule part, said capsule being openable by heating said at least one heating element under an effect of at least one alternating magnetic field, said capsule dissolving when entering in contact with a solving liquid.

21. A capsule according to claim 20, wherein the at least one heating element includes a mixture of a magnetic powder and an evaporating liquid.

22. A capsule according to claim 21, wherein the at least one heating element includes a mixture of about 40 Vol-% magnetite and 60 Vol-% ethyl alcohol.

23. A capsule according to claim 20, wherein said heating element includes a liquid containing substance contained in a bag, a volume of which is expandable to at least double a non-inflated size when the liquid is evaporating.

24. A capsule according to claim 21, wherein the mixture is contained in a bag, a volume of which is expandable to at least double a non-inflated size when the liquid is evaporating.

25. A capsule according to claim 22, wherein the mixture is contained in a bag, a volume of which is expandable to at least double a non-inflated size when the liquid is evaporating.

26. A capsule according to claim 20, wherein said other of said capsule parts is provided with at least one opening through which the at least one heated heating element presses the agent to the outside and the solving liquid enters the capsule.

27. A capsule according to claim 26, wherein the solving liquid is water.

28. A capsule according to claim 20, wherein said capsule parts are surrounded by a protective foil against the solving liquid.

29. A capsule according to claim 20, wherein the capsule parts are partly nested one into an other.

30. A capsule according to claim 28, wherein:
the capsule parts are partly nested one into an other; and
the capsule parts have common contact surfaces that are not covered by the protective foil.

31. A capsule according to claim 30, wherein the common contact surfaces are provided with a lubricant.

32. A capsule according to claim 20, wherein the heating element acts on the other of said capsule parts via a stamp.

33. A capsule according to claims 21, wherein the heating element acts on the other of said capsule parts via a stamp.

34. A capsule according to claims 22, wherein the heating element acts on the other of said capsule parts via a stamp.

35. A capsule according to claim 32, wherein the insulating capsule part that surrounds the heating element is at least partially provided with guide surfaces for the stamp.

36. A capsule according to claim 33, wherein the insulating capsule part that surrounds the heating element is at least partially provided with guide surfaces for the stamp.

37. A capsule according to claim 34, wherein the insulating capsule part that surrounds the heating element is at least partially provided with guide surfaces for the stamp.

38. A capsule according to claim 32, wherein the insulating capsule part and the other of said capsule parts are lifted one from an other when the heating element is heated.

39. A capsule according to claim 33, wherein the insulating capsule part and the other of said capsule parts are lifted one from an other when the heating element is heated.

40. A capsule according to claim 34, wherein the insulating capsule part and the other of said capsule parts are lifted one from an other when the heating element is heated.

41. A capsule according to claim 20, further comprising a movable and/or expandable wall being disposed between the heating element and the agent.

42. A capsule according to claim 20, wherein the material with the greater thermal resistance includes air or another gas that is located between two walls made of hard gelatin.

43. A capsule according to claim 20, wherein the material with the greater thermal resistance includes a water-soluble porous substance.

44. A capsule according to claim 20, wherein said at least one heating element includes two heating elements contained in a diametral arrangement, each being surrounded by a corresponding one of said at least one insulating capsule part, which adjoin each other.

45. A capsule according to claim 20, wherein said at least one heating element includes at least two heating elements comprised of different materials or mixtures.

46. A capsule according to claim 44, wherein said two heating elements are comprised of different materials or mixtures.

47. A capsule according to claim 20, wherein said at least one agent includes different agents that can be mixed with each other.

48. A capsule according to claim 44, wherein said at least one agent includes different agents that can be mixed with each other.

49. A capsule for releasing at least one agent contained therein at defined positions in a body, comprising:
capsule parts enclosing said at least one agent in said capsule; and
at least one heating element at least partially surrounded by a thermally insulating envelope, said capsule being openable by expansion caused by heating said at least one heating element under an effect of at least one alternating magnetic field.

50. A capsule according to claim 49, wherein a thermal resistance of the thermally insulating envelope is about 500 K/W.
